SEQUENCE LISTING (4 sequences)

SEQ ID NO: 1 - DNA, 2679 bp, Homo Sapien
ggcgggcgcgccgggcggcaggtgtcggcgtcggcggcattcggcggcgatggagcggccctggggagctgcggacggcctctcgcgctggccccatggcctcggcctcctcctcctcctgcagctgctgccgccgtcgaccctcagccaggaccggctggacgcgccgccgccgcccgctgcgccgctgccgcgctggtctggccccatcggggtgagctgggggctgcgggcggccgcagccgggggcgcgtttccccgcggcggccgttggcgtcgcagcgcgccgggcgaggacgaggagtgcggccgggtccgggacttcgtcgccaagctggccaacaacacgcaccagcatgtgtttgatgatctcagaggctcagtatccttgtcctgggttggagatagcactggggtcattctagtcttgactaccttccatgtaccactggtaattatgacttttggacagtccaagctatatcgaagtgaggattatgggaagaactttaaggatattacagatctcatcaataacacctttattcggactgaatttggcatggctattggtcctgagaactctggaaaggtggtgttaacagcagaggtgtctggaggaagtcgtggaggaagaatcttcagatcatcagattttgcgaagaattttgtgcaaacagatctcccttttcatcctctcactcagatgatgtatagccctcagaattctgattatcttttagctctcagcactgaaaatggcctgtgggtgtccaagaattttgggggaaaatgggaagaaatccacaaagcagtatgtttggccaaatggggatcagacaacaccatcttctttacaacctatgcaaatggctcctgcaaagctgaccttggggctctggaattatggagaacttcagacttgggaaaaagcttcaaaactattggtgtgaaaatctactcatttggtcttgggggacgtttcctttttgcctctgtgatggctgataaggataccacaagaaggatccacgtttcaacagatcaaggggacacatggagcatggcccagctcccctccgtgggacaggaacagttctattctattctggcagcaaatgatgacatggtattcatgcatgtagatgaacctggagacactgggtttggcacaatctttacctcagatgatcgaggcattgtctattccaagtctttggaccgacatctctacactaccacaggcggagagacggactttaccaacgtgacctccctccgcggcgtctacataacaagcgtgctctccgaagataattctatccagaccatgatcacttttgaccaaggaggaaggtggacgcacctgaggaagcctgaaaacagtgaatgtgatgctacagcaaaaaacaagaatgagtgcagccttcatattcatgcttcctacagcatctcccagaaactgaatgttccaatggccccactctcagagccgaatgccgtaggcattgtcattgctcatggtagcgtgggggatgccatctcagtgatggttccagatgtgtacatctcagatgatgggggttactcctggacaaagatgctggaaggaccccactattacaccatcctggattctggaggcatcattgtggccattgagcacagcagccgtcctatcaatgtgattaagttctccacagacgaaggtcaatgctggcaaacctacacgttcaccagggaccccatctatttcactggcctagcttcagaacctggagctaggtccatgaatatcagcatttggggcttcacagaatctttcctgaccagccagtgggtctcctacaccattgattttaaagatatccttgaaaggaactgtgaagagaaggactataccatatggctggcacactccacagaccctgaagattatgaagatggctgcattttgggctacaaagaacagtttctgcggctacgcaagtcatccgtgtgtcagaatggtcgagactatgttgtgaccaagcagccctccatctgcctctgttccctggaggactttctctgtgattttggctactaccgtccagaaaatgactccaagtgtgtggaacagccagaactgaagggccacgacctggagttttgtctgtacggaagagaagaacacctaacaacaaatgggtaccggaaaattccaggggacaaatgccagggtggggtaaatccagttcgagaagtaaaagacttgaaaaagaaatgcacaagcaactttttgagtccggaaaaacagaattccaagtcaaattctgttccaattatcctggccatcgtgggattgatgctggtcacagtcgtagcaggagtgctcattgtgaagaaatatgtctgtgggggaaggttcctggtgcatcgatactctgtgctgcagcagcatgcagaggccaatggtgtggatggtgtggatgctttggacacagcctcccacactaataaaagtggttatcatgatgactcagatgaggacctcttggaatagctcttcagaggagctggacccagcatggatggtggaaccacagtacctcttacactccctgtggctccaacttcaggaaataaatttcccattgcgagggaaaaaaaaaaaaaaaaaaaaaaaaaaaaaaaaaa

SEQ ID NO: 2 - protein, 831 aa, Homo Sapien
MERPWGAADGLSRWPHGLGLLLLLQLLPPSTLSQDRLDAPPPPAAPLPRWSGPIGVSWGLRAAAAGGAFPRGGRWRRSAPGEDEECGRVRDFVAKLANNTHQHVFDDLRGSVSLSWVGDSTGVILVLTTFHVPLVIMTFGQSKLYRSEDYGKNFKDITDLINNTFIRTEFGMAIGPENSGKVVLTAEVSGGSRGGRIFRSSDFAKNFVQTDLPFHPLTQMMYSPQNSDYLLALSTENGLWVSKNFGGKWEEIHKAVCLAKWGSDNTIFFTTYANGSCKADLGALELWRTSDLGKSFKTIGVKIYSFGLGGRFLFASVMADKDTTRRIHVSTDQGDTWSMAQLPSVGQEQFYSILAANDDMVFMHVDEPGDTGFGTIFTSDDRGIVYSKSLDRHLYTTTGGETDFTNVTSLRGVYITSVLSEDNSIQTMITFDQGGRWTHLRKPENSECDATAKNKNECSLHIHASYSISQKLNVPMAPLSEPNAVGIVIAHGSVGDAISVMVPDVYISDDGGYSWTKMLEGPHYYTILDSGGIIVAIEHSSRPINVIKFSTDEGQCWQTYTFTRDPIYFTGLASEPGARSMNISIWGFTESFLTSQWVSYTIDFKDILERNCEEKDYTIWLAHSTDPEDYEDGCILGYKEQFLRLRKSSVCQNGRDYVVTKQPSICLCSLEDFLCDFGYYRPENDSKCVEQPELKGHDLEFCLYGREEHLTTNGYRKIPGDKCQGGVNPVREVKDLKKKCTSNFLSPEKQNSKSNSVPIILAIVGLMLVTVVAGVLIVKKYVCGGRFLVHRYSVLQQHAEANGVDGVDALDTASHTNKSGYHDDSDEDLLE

SEQ ID NO: 3 - protein, 42 aa, Homo Sapien [sequence omitted]

SEQ ID NO: 4 - protein, 8 aa, Homo Sapien
DYKDDDDK

(19) United States
(12) Patent Application Publication
(10) Pub. No.: US 2010/0028333 A1
(43) Pub. Date: Feb. 4, 2010

(54) RECEPTOR FOR AMYLOID BETA AND USES THEREOF

(76) Inventors: Krista L. Getty, Quakertown, PA (US); William J. Ray, Lansdale, PA (US); Cloud P. Paweletz, Boston, MA (US)

Correspondence Address: MERCK AND CO., INC, P O BOX 2000, RAHWAY, NJ, 07065-0907, US

(21) Appl. No.: 12/519,249

(22) PCT Filed: Dec. 11, 2007
(86) PCT No.: PCT/US07/25331

§ 371 Date: Jun. 15, 2009

Related U.S. Application Data
(60) Provisional application No. 60/875,046, filed on Dec. 15, 2006.

(51) Int. Cl. A61K 39/395; G01N 33/53

(52) U.S. Cl. 424/130.1; 435/7.1

(57) ABSTRACT

Compositions and methods for identifying modulators of sortilin are described. The methods are particularly useful for identifying analytes that antagonize sortilin s effect on processing of amyloid precursor protein to Aβ peptide and thus useful for identifying analytes that can be used for treating Alzheimer disease.

### CROSS REFERENCE TO RELATED APPLICATIONS

This application claims the benefit of U.S. Provisional Application No. 60/875,046, filed Dec. 15, 2006, the contents of which are incorporated herein by reference in their entirety.

### BACKGROUND OF THE INVENTION

(1) Field of the Invention

The present invention relates to the use of sortilin as a receptor for amyloid beta and uses thereof. The methods disclosed herein are particularly useful for identifying analytes that modulate sortilin's interaction with amyloid beta and thus useful for identifying analytes that can be used for preventing and treating Alzheimer disease.

(2) Description of Related Art

Alzheimer's disease (AD) is a common, chronic neurodegenerative disease, characterized by a progressive loss of memory and behavioral abnormalities, as well as an impairment of other cognitive functions that often leads to dementia and death. AD ranks as the fourth leading cause of death in industrialized societies after heart disease, cancer, and stroke. The incidence of Alzheimer's disease is high, with an estimated 4.5 million patients affected in the United States and perhaps 17 to 25 million worldwide. Moreover, the number of sufferers is expected to grow as the population ages.

A characteristic feature of Alzheimer's disease is the presence of large numbers of insoluble deposits, known as amyloid plaques, in the brains of those affected (Cummings & Cotman, Lancet 326:1524-1587 (1995)). The most widely held hypothesis in the AD field is that amyloid plaques and/or soluble aggregates of amyloid peptides are intimately, and perhaps causally, involved in Alzheimer's disease.

A variety of experimental evidence supports this view. For example, amyloid β (Aβ) peptide, the primary proteinaceous component of amyloid plaques, is toxic to neurons in culture and transgenic mice that overproduce Aβ peptide in their brains show extensive deposition of Aβ into amyloid plaques (Yankner, Science 250:279-282 (1990); Mattson et al., J. Neurosci. 12:379-389 (1992); Games et al., Nature 373:523-527 (1995); LaFerla et al., Nature Genetics 9:21-29 (1995)). Mutations in the APP gene leading to increased Aβ production cause heritable forms of Alzheimer's disease (Goate et al., Nature 349:704-706 (1991); Chartier-Harlan et al., Nature 353:844-846 (1991); Murrel et al., Science 254:97-99 (1991); Mullan et al., Nature Genetics 1:345-347 (1992)). Presenilin-1 (PS1) and presenilin-2 (PS2)-related familial early-onset Alzheimer's disease (FAD) are associated with disproportionately increased production of Aβ1-42, the 42 amino acid isoform of Aβ, as opposed to Aβ1-40, the 40 amino acid isoform (Scheuner et al, Nature Medicine 2:864-870 (1996)). This longer 42 amino acid isoform of Aβ is more prone to aggregation than the shorter isoform (Jarrett et al, Biochemistry 32:4693-4697 (1993). Injection of the insoluble, fibrillar form of Aβ into monkey brains results in the development of pathology (neuronal destruction, tau phosphorylation, microglial proliferation) that mimics Alzheimer's disease in humans (Geula et al., Nature Medicine 4:827-831 (1998)). See Selkoe, J., Neuropathol. Exp. Neurol. 53:438-447 (1994) for a review of the evidence that Aβ has a central role in Alzheimer's disease.

Aβ peptide is a 39-43 amino acid peptide derived by proteolytic cleavage of the amyloid precursor protein (APP). APP is membrane bound and undergoes proteolytic cleavage by at least two pathways. In one pathway, cleavage by an enzyme known as α-secretase occurs (Kuentzel et al., Biochem. J. 295:367-378 (1993)). This cleavage by α-secretase occurs within the Aβ peptide portion of APP, thus precluding the formation of Aβ peptide. In another proteolytic pathway, cleavage of the Met596-Asp597 bond (numbered according to the 695 amino acid protein) by β-secretase occurs. This cleavage by β-secretase generates the N-terminus of Aβ peptide. The C-terminus is formed by cleavage by γ-secretase. The C-terminus is actually a heterogeneous collection of cleavage sites rather than a single site since γ-secretase activity occurs over a short stretch of Aβ amino acids rather than at a single peptide bond. Peptides of 40 or 42 amino acids in length (Aβ40 and Aβ42, respectively) predominate among the C-termini generated by γ-secretase. Aβ42 peptide is more prone to aggregation than Aβ40 peptide (Jarrett et al., Biochemistry 32: 4693-4697 91993); Kuo et al., J. Biol. Chem. 271:4077-4081 (1996)), and its production is closely associated with the development of Alzheimer's disease (Sinha and Lieberburg, Proc. Natl. Acad. Sci. USA 96:11049-11053 (1999)). The bond cleaved by γ-secretase appears to be situated within the transmembrane domain of Aβ. For a review that discusses Aβ and its processing, see Selkoe, Trends Cell. Biol. 8:447-453 (1998).

Additional studies have focused on the possibility that extracellular oligomers of Aβ, such as Aβ derived diffusible ligands (“ADDLs”) impair physiological processes involved in learning and memory (Walsh et al., Neuron 44(1):181-193 (2004)) and, as such, are the principal agents believed to be responsible for the neuropathology of Alzheimer's disease.

Currently, many therapeutic strategies focused on modifying the pathology of Alzheimer's disease have targeted the secretase proteins directly responsible for the processing of Aβ from APP or modulators of Aβ formation or secretion. Secretase inhibitors have been plagued either by mechanism-based toxicity (γ-secretase inhibitors), γ-secretase cleaves many substrates including the key signaling molecule Notch, or by difficulties in identifying small molecule inhibitors with appropriate pharmacokinetic properties to allow them to become drugs (BACE inhibitors). For a review of the issues associated with inhibiting secretases for AD therapy see Beher D, et al., Expert Opin. Investig. Drugs 11:1385-1409 (2005). Another strategy recently proposed is the removal of Aβ from the circulation or the brain by passive or active immunization against the Aβ peptide (reviewed in Schenk D B, et al., Neurodegener. Dis. 2(5):255-260 (2005)). However, these approaches also have limitations, such as whether large numbers of people will safely tolerate active immunization against a naturally occurring self-generated peptide. Still another therapeutic strategy is to block the effects of Aβ on brain cells by interfering with its ability to interact with specific proteins. This strategy has not been tested as yet because little is known about the neuronal proteins that are important for Aβ toxicity, notwithstanding that this area has been extensively studied (reviewed in Smith W W, et al., CNS Neurol. Disord. Drug Targets 5(3):355-361 (2006)). The present invention provides methods for identifying new treatments for Alzheimer's disease by modulating the interaction between Aβ and sortilin, a protein expressed in brain cells.

### BRIEF SUMMARY OF THE INVENTION

The present invention provides methods for identifying analytes that modulate the interaction of sortilin and Aβ. The methods are particularly useful for identifying analytes that antagonize sortilin's ability to bind to the Aβ peptide and, thus, useful for identifying analytes that can be used for preventing or treating Alzheimer disease.

Therefore in one embodiment the present invention provides a nucleotide sequence (SEQ ID NO:1) of an isolated human cDNA encoding a human sortilin polypeptide as shown in SEQ ID NO:2 complexed with Aβ (SEQ ID NO: 3) and recombinant cell lines expressing said complex for use in the methods herein. Sortilin was identified by biochemically purifying receptors for Aβ in mammalian brain extract as set forth in Example 1.

In another embodiment, the present invention provides a method for screening for analytes that antagonize the binding of sortilin to Aβ peptide, comprising providing cells that express sortilin AD; incubating the cells in a culture medium containing synthetic, natural, or labeled Aβ either in monomeric, oligomeric, or fibrillar form, and which contains an analyte; removing the culture medium from the recombinant cells; and determining the amount Aβ bound to cells, internalized within cells, or depleted from the medium by the sortilin-expressing cells, and determining additionally if the analyte inhibited Aβ binding, internalization, or depletion. The invention can also be used to screen and/or identify other components that contribute to Aβ's toxicity.

In further aspects of the method, the recombinant cells each comprise a first nucleic acid that encodes sortilin operably linked to a first heterologous promoter. In preferred aspects of the present invention, the Aβ is synthetically prepared with a fluorescent label, aggregated into oligomers, and incubated with sortilin expressing cells. In preferred aspects, the method includes a control which comprises providing recombinant cells incubated with Aβ that do not express sortilin.

In light of the analytes that can be identified using the above methods, the present invention further provides a method for treating Alzheimer's disease in an individual which comprises providing to the individual an effective amount of an antagonist of sortilin amyloid binding activity.

Further still, the present invention provides a method for identifying an individual who has Alzheimer's disease or is at risk of developing Alzheimer's disease comprising obtaining a sample from the individual and measuring the amount of sortilin complexed with Aβ in the sample.

Further still, the present invention provides for the use of an antagonist of sortilin for the manufacture of a medicament for the treatment of Alzheimer's disease.

Further still, the present invention provides for the use of an antibody that disrupts or prevents the complex between sortilin and Aβ for the manufacture of a medicament for the treatment of Alzheimer's disease.

Further still, the present invention provides a vaccine for preventing and/or treating Alzheimer's disease in a subject, comprising an antibody raised against an antigenic amount of sortilin wherein the antibody antagonizes the interaction of sortilin to Aβ peptide.

The term “analyte” refers to a compound, chemical, agent, composition, antibody, peptide, aptamer, nucleic acid, or the like, which can modulate the activity of sortilin.

The term “sortilin” refers to a cell surface receptor that is a member of the vacuolar protein sorting 10 domain (Vps10p-D) receptor family. Sortilin is believed to be involved in membrane trafficking and transport of proteins to the endosomal/lysosomal system (Nielsen M S, et al., EMBO J. 20(9):2180-2190 (2001)). The sortilin gene encodes an 833 amino acid protein (NP_{—}002950). The encoded protein, a transmembrane protein that is a type-I receptor, binds a number of unrelated ligands that participate in a wide range of cellular processes, but lacks the typical features of a signaling receptor. The nucleotide sequence is reported as Genbank ID number BC023542. The term further includes mutants, variants, alleles, and polymorphs of sortilin. Where appropriate, the term further includes fusion proteins comprising all or a portion of the amino acid sequence of sortilin fused to the amino acid sequence of a heterologous peptide or polypeptide, for example, hybrid immuoglobulins comprising the amino acid sequence, or domains thereof, of sortilin fused at its C-terminus to the N-terminus of an immunoglobulin constant region amino acid sequence (see, for example, U.S. Pat. No. 5,428,130 and related patents).

The term “sortilin derivative” or “derivatives” refers to a polypeptide or protein produced from a cDNA that encodes a part or all of the sortilin sequence, or a polypeptide or protein produced from purified sortilin, including polypeptides or proteins that have been modified by altering the primary cDNA coding sequence or by introducing biochemical alterations to the purified native sortilin.

The term “sortilin fragment” or “fragments” refers to naturally occurring or synthetically produced portions of the sortilin protein.

### BRIEF DESCRIPTION OF THE DRAWINGS

FIG. 1 is a nucleic sequence encoding the human sortilin (SEQ ID NO:1).

FIG. 2 is the amino acid sequence of the human sortilin (SEQ ID NO:2).

FIG. 3 shows the binding of ADDLs to primary hippocampal neurons.

FIG. 4A is a graphic depicting the method for identifying sortilin as an ADDL receptor: SA—streptavidin; EGS—ethylene glycol-bis-succinimidyl succinate; bADDL (EV) 1-42—biotinylated ADDLs. FIG. 4B shows the identification of sortilin as a receptor for Aβ: lane 1—molecular weight marker; lane 4 cerebellum (proteins cross linked to ADDL); lane 5—hippocampus (proteins cross linked to ADDL). FIG. 4C is a western blot of ADDL-precipitated proteins: C—cerebellum; H—hippocampus; Brain hmgt—brain homogentate/total membranes from indicated region; Supt—proteins not recovered by streptavidin beads; Pellet—proteins associated with streptavidin beads; B103 and CHO—lysates from cell lines with high (B103) and low (CHO) ADDL binding.

FIG. 5 shows the physical interaction between sortilin and ADDLs by immunoprecipitation (IP): IB—immunoblot; sort-sortilin.

FIG. 6 shows the localization of sortilin protein with amyloid plaques in transgenic mice.

FIG. 7 shows the effect of sortilin overexpression on Aβ 40 levels in cell culture medium.

FIG. 8 shows the tissue distribution of sortilin mRNA in various human tissues.

### DETAILED DESCRIPTION OF THE INVENTION

Applicants herein have found that a previously known protein, sortilin, is a receptor for Aβ and that antagonists or modulators of sortilin can be used to modulate its binding or interaction with Aβ.

Sortilin (also known as gp95) has been identified as a receptor-associated protein (RAP)-binding protein. Whereas RAP is an endoplasmic reticulum/Golgi protein involved in the processing of receptors of the low density lipoprotein receptor family (Petersen et al., J. Biol. Chem. 272 (6):3599-3605 (1997)), sortilin is expressed in brain, spinal cord and testis and has homology to established sorting receptors. Mazella et al., J. Biol. Chem. 273 (41): 26273-26276 (1998) cloned a neurotensin (NT) receptor that was identical to the previously identified gp95/sortilin (that was purified from human brain). Sortilin is a cell surface receptor of the vacuolar protein sorting 10 domain (Vps10p-D) receptor family, which includes SorLA (also known as LR11), which is found to be decreased in AD patients perhaps leading to an increase in extracellular Aβ levels, (Scherzer et al., Arch. Neurol. 61:2001205 (2004)), and S or CS1-3. Sortilin is involved in membrane trafficking and transport of proteins to the endosomal/lysosomal system, a known site of Aβ42 accumulation in neurons of AD patients (Gouras et al., Neurobiology of Aping 26: 235-1244 (2005)). Sortilin complexes with p75^{NTR} on the cell surface and acts as a co-receptor for proNGF and proBDNF and is responsible for inducing neuronal death (Teng et al., J. Neuroscience 25(22):5455-5463 (2005)). p75^{NTR} is also believed to play a role in binding Aβ (Yaar et al., J. Clinical Investigation 100(9):2333-2340 (1997)). Furthermore, sortilin is a receptor for apolipoprotein E, the major genetic risk factor for AD (Beffert and Poirier, Ann. N.Y. Acad. Sci. 777:166-174 (1996)).

ADDLs bind specifically to primary hippocampal neurons in vitro creating a punctate binding pattern characteristic of a cell-surface receptor binding event (Lacor et al., 2004, and in Klein W L, et al., Neurobiol. Aging 25(5):569-580 (2004)). The molecular species expressed in neurons that mediate this binding are not known. This finding prompted Applicants to identify potential receptor(s) on the cell surface of the neurons that are binding Aβ/ADDLs in order to inhibit binding and, thus, Aβ toxicity to neurons.

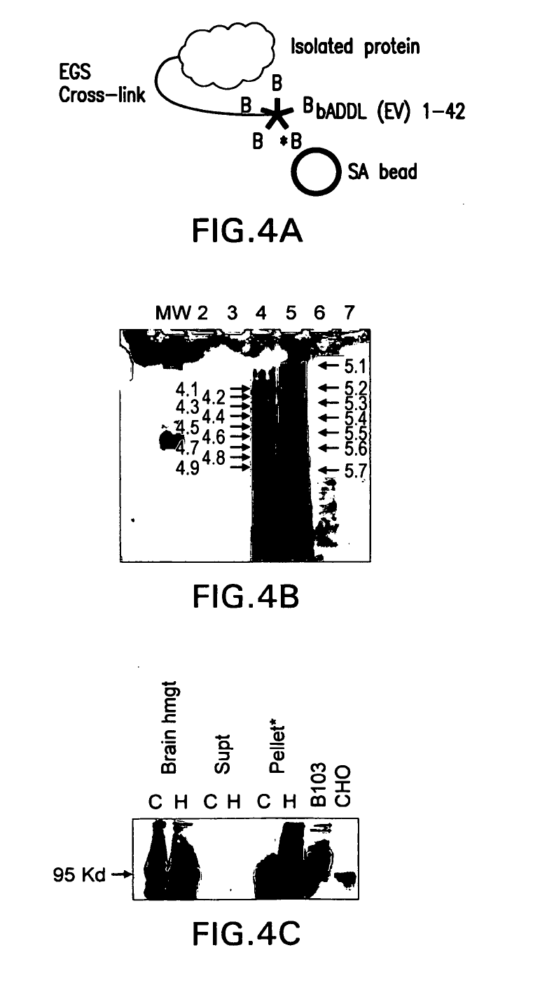

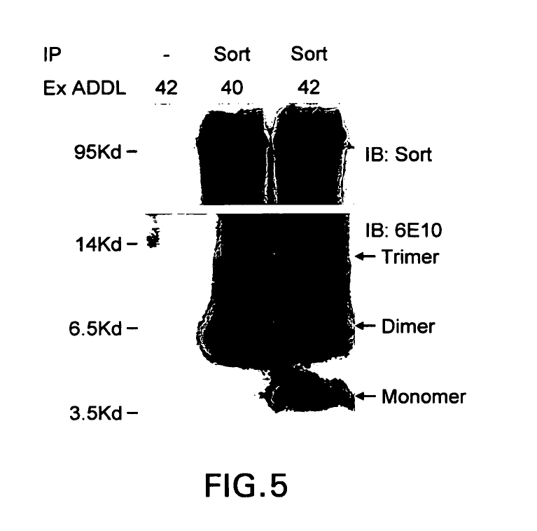

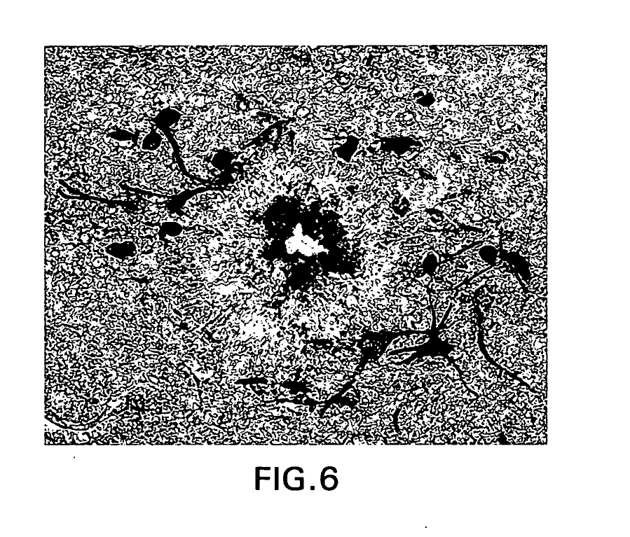

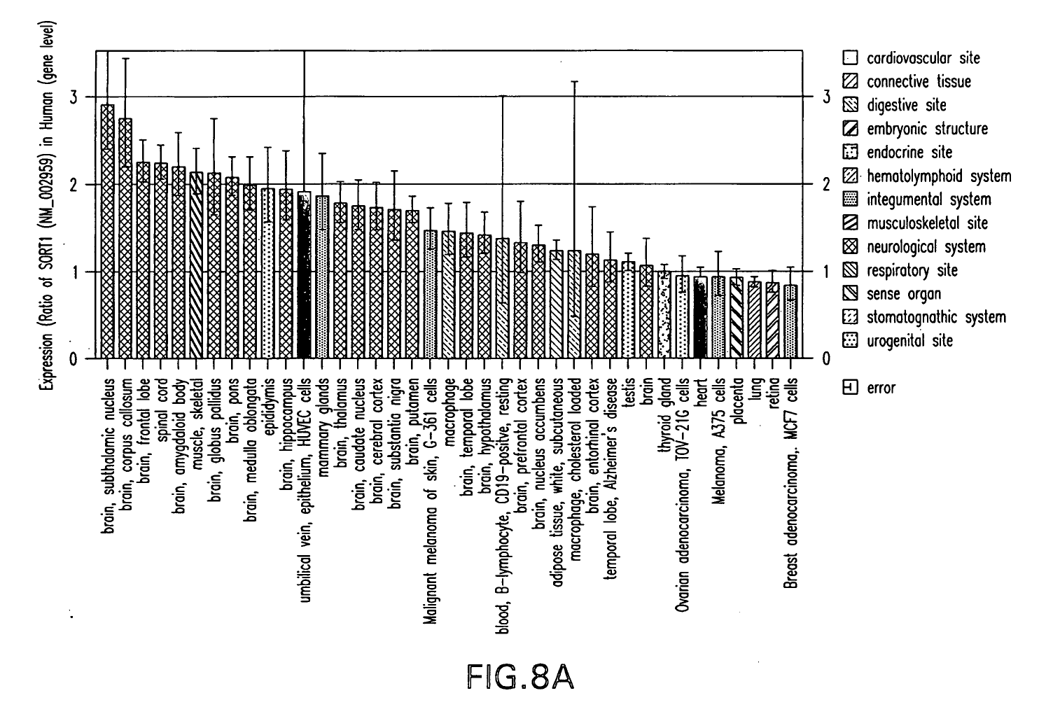

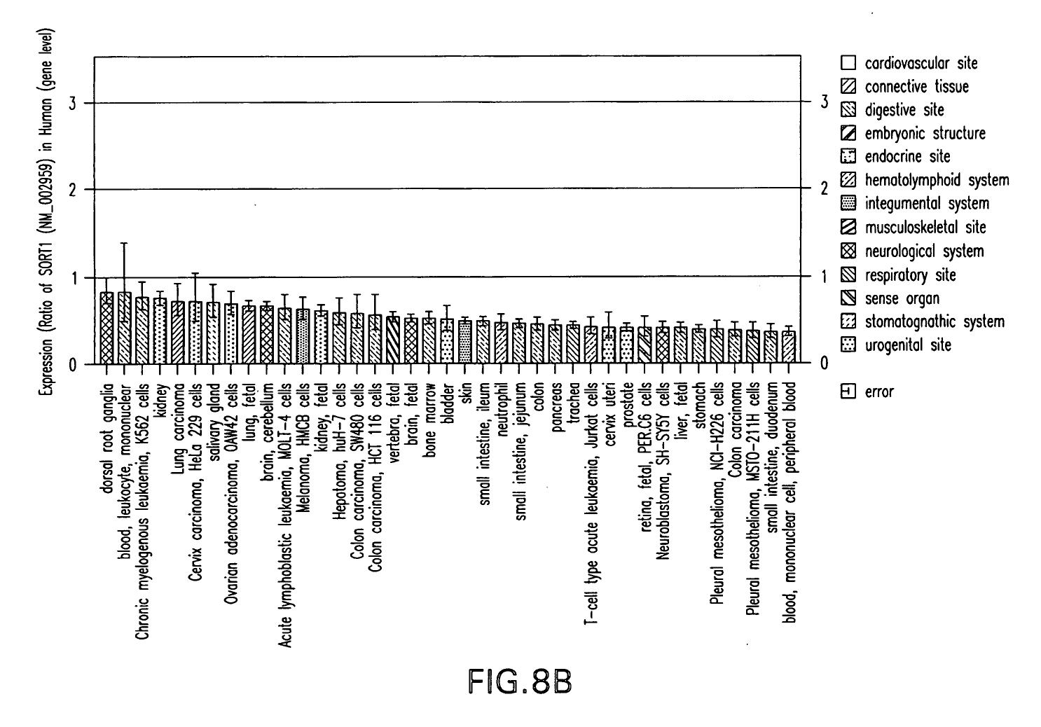

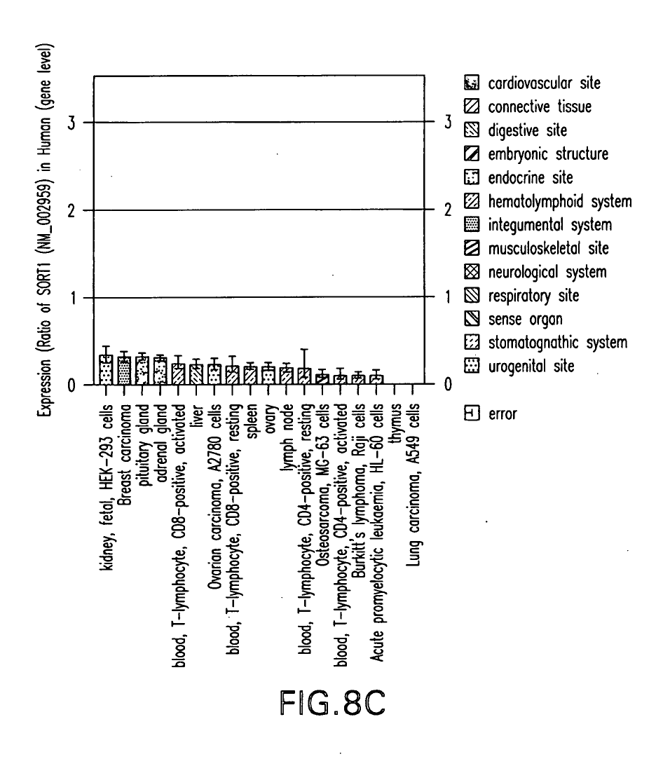

To identify the putative Aβ receptor, ADDLs prepared from biotinylated Aβ42 were used as “bait” in a cross-linking immunoprecipitation experiment (schematically shown in FIG. 4A) performed on membrane preparations isolated from either rat hippocampus and cerebellum (used as a control in that AD pathology is not observed in this part of the brain). ADDLs were incubated with membrane proteins prepared from these brain regions to allow binding to receptors, and chemical cross-linking was used to stabilize the ADDL-receptor complexes. The ADDL-receptor complexes were precipitated with streptavidin coated beads, which bind the biotin incorporated into the synthetic ADDLs. The chemical cross-links were then broken and the proteins that had been recovered from both hippocampus and cerebellum were separated on an SDS-PAGE gel. Groups of proteins were extracted from the gel and analyzed by trypsin digestion followed by mass spectrometry. One of the resulting proteins identified by Applicants herein was sortilin, which was found in the proteins recovered from the hippocampus but much less abundantly recovered from cerebellum. As sortilin is a receptor-like protein expressed prominently in the brain (FIG. 8), Applicants reasoned that sortilin could be a putative Aβ receptor. Additional biochemical experiments confirmed that Aβ peptides exist in a complex with sortilin (FIG. 5). In this latter experiment, Aβ40 or Aβ42 was added to the cell culture medium of HEK293 cells, which express and secrete sortilin. Immunoprecipitation of secreted sortilin from the culture media by anti-sortilin antibodies recovers monomers and multimers of both Aβ40 and Aβ42, indicating that sortilin-Aβ complexes had formed. Furthermore, cDNA overexpression of sortilin protein produces a reduction in Aβ levels in the medium of cultured HEK293 cells overexpressing APP_{NFEV} (FIG. 6). This data is consistent with enhanced receptor-mediated internalization and degradation of Aβ in sortilin over-expressing cells.

To demonstrate the relevance of the sortilin-Aβ interaction to the disease state, sortilin protein localization was examined in the brains of mice that had developed amyloid plaques. As shown in FIG. 6, immunohistochemical staining of brain sections shows that sortilin protein accumulates in neuronal and glial cells adjacent to amyloid deposits. Immunoreactive areas stain dark where sortilin protein is expressed. These data clearly show cells appearing to be microglial and astrocytic cells near the plaque darkly staining for sortilin. Additionally, dystrophic neurites appear as long thin rod-like structures and stain positive for sortilin. Consistent with a protein that binds Aβ, sortilin immunoreactivity localizes to most amyloid plaques. In this representative figure, sortilin immunoreactivity is strongest in the core of the plaque. These data demonstrate that sortilin accumulates in cells adjacent to high concentrations of Aβ in an animal model of Alzheimer's disease, and furthermore that sortilin accumulates within amyloid plaques. Together these data show 1) sortilin binds Aβ, 2) mediates its uptake into cells, and 3) accumulates in cells near amyloid plaques. Thus, inhibiting sortilin binding to Aβ would provide therapeutic benefit in AD patients by promoting the clearance of Aβ and/or preventing its internalization into neural cells.

Notwithstanding that sortilin has never been suggested to be a receptor for Aβ and, is thus, a novel target for abrogating amyloid toxicity, published data interpreted within the context of Applicants discovery supports the conclusion that sortilin is a potential therapeutic target for AD. As described above, sortilin is a co-receptor for proNGF, a peptide produced in neural tissue in response to injury and in AD that causes cell death (references above). Sortilin binds proNGF with p75^{NTR}, which is also an independent protein receptor for AD. Furthermore sortilin is a substrate for γ-secretase (Nyborg A C, et al., Mol. Neurodegener. 1:3 (2006)). Inhibitors of sortilin designed to block interaction with proNGF, a protein unrelated to Aβ, have been claimed (WO2005044293 A3). Additionally, modulators of sortilin and its related proteins for the enhancement of neurotrophin signaling, a process unrelated to Aβ, have also been claimed (WO2004056385 A2).

Sortilin can be targeted as a therapeutic for AD in a number of ways. Sortilin or derivatives (defined above) can be injected into AD patients in order to bind and neutralize Aβ. In this therapy, sortilin or its derivative or fragment will be produced under conditions that allow it to be collected at high purity yet retain high affinity for Aβ when produced in isolation. An effective amount of this product is then injected into the patient with AD. This product then complexes with and neutralizes Aβ, thereby providing therapy to the patient. Sortilin expression could be reduced in the brain by using silencing RNA or other techniques. In this therapy, an siRNA or another gene silencing agent (such as an shRNA) is introduced into the patient with AD at effective doses and in a manner that allows the siRNA to enter the brain. The siRNA then reduces the expression of sortilin mRNA and thereby provides a therapeutic benefit to the patient. Likewise analytes that interfere with the sortilin-Aβ interaction or with sortilin trafficking to the cell surface or from the cell surface to the endosomal system can be administered to AD patients.

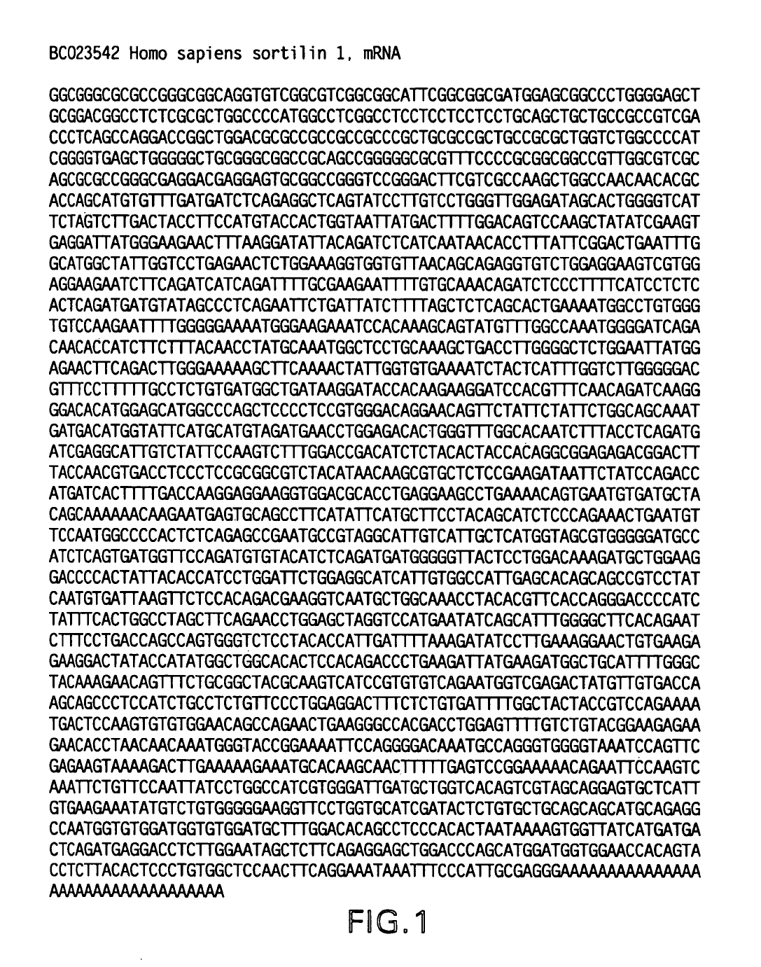

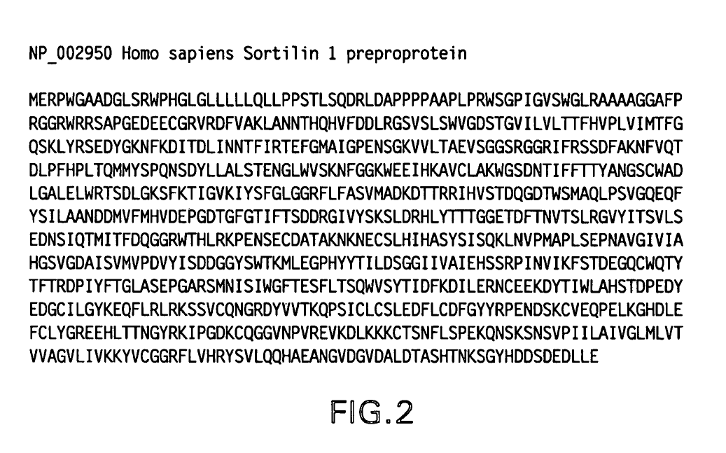

The nucleic acid sequence encoding human sortilin (SEQ ID NO:1) is shown in FIG. 1 and the amino acid sequence for human sortilin (SEQ ID NO:2) is shown in FIG. 2. The amino acid sequence for human Aβ peptide is known, DAEFRHDSGYEVHHQKLVFFAED VGSNKGAIIGLMVGGVVIA (SEQ ID NO:3) (Kang J, et al., Nature 325:733-736 (1987).

The mRNA encoding sortilin was found to be preferentially enriched in regions of the brain subject to Alzheimer's disease pathology (FIG. 8).

In light of applicants' discovery, sortilin, or its derivative, as set forth in Examples 1-5 is useful for identifying analytes which antagonize its interaction with Aβ. These analytes can be used to treat patients afflicted with Alzheimer's disease. Sortilin-based therapies will be used alone or in combination with acetylcholinesterase inhibitors, NMDA receptor partial agonists, secretase inhibitors, amyloid-reactive antibodies, and other treatments for Alzheimer's disease.

The present invention provides methods for identifying sortilin modulators by contacting sortilin with a substance that inhibits or stimulates sortilin expression and determining whether expression of sortilin polypeptide or nucleic acid molecules encoding sortilin are modified. The present invention also provides methods for identifying modulators that antagonize sortilin's effect on its interaction with Aβ peptide in tissues where sortilin is localized or co-expressed. For example, sortilin protein can be expressed in cell lines that produce, express, or are incubated with Aβ and the effect of the modulator on the interaction of sortilin and Aβ (s-Aβ) is monitored using standard biochemical assays with Aβ-specific antibodies or by mass spectrophotometric techniques. Inhibitors for the s-Aβ interaction are identified by screening for changes in the cytotoxicity or cell surface binding of Aβ as exemplified in Example 7. Both small molecules and larger biomolecules that antagonize sortilin-mediated interaction with Aβ peptide can be identified using such an assay. A method for identifying antagonists of sortilin's effect on the s-Aβ interaction includes the methods herein which are amenable to high throughput screening. In addition, the methods disclosed in U.S. Pub. Pat. Appln. No. 20030200555 can be adapted to use in assays for identifying antagonists of sortilin activity.

A mammalian sortilin cDNA, encompassing the first through the last predicted codon contiguously, is amplified from brain total RNA with sequence-specific primers by reverse-transcription polymerase chain reaction (RT-PCR). The amplified sequence is cloned into pcDNA3.zeo or other appropriate mammalian expression vector. Fidelity of the sequence and the ability of the plasmid to encode full-length sortilin is validated by DNA sequencing of the sortilin plasmid (pcDNA_sortilin).

Commercially available mammalian expression vectors which are suitable for recombinant sortilin expression include, but are not limited to, pcDNA3.neo (Invitrogen, Carlsbad, Calif.), pcDNA3.1 (Invitrogen, Carlsbad, Calif.), pcDNA3.1/Myc-His (Invitrogen), pCI-neo (Promega, Madison, Wis.), pLITMUS28, pLITMUS29, pLITMUS38 and pLITMUS39 (New England Biolabs, Beverly, Mass.), pcDNAI, pcDNAIamp (Invitrogen), pcDNA3 (Invitrogen), pMC1neo (Stratagene, La Jolla, Calif.), pXT1 (Stratagene), pSG5 (Stratagene), EBO-pSV2-neo (ATCC 37593) pBPV-1(8-2) (ATCC 371.10), pdBPV-MMTneo (342-12) (ATCC 37224), pRSVgpt (ATCC 37199), pRSVneo (ATCC 37198), pSV2-dhfr (ATCC 37146), pUCTag (ATCC 37460), lZD35 (ATCC 37565), pMC1neo (Stratagene), pcDNA3.1, pCR3.1 (Invitrogen, San Diego, Calif.), EBO-pSV2-neo (ATCC 37593), pCI.neo (Promega), pTRE (Clontech, Palo Alto, Calif.), pV1Jneo, pIRESneo (Clontech, Palo Alto, Calif.), pCEP4 (Invitrogen,), pSC11, and pSV2-dhfr (ATCC 37146). The choice of vector will depend upon the cell type in which it is desired to express the sortilin, as well as on the level of expression desired, cotransfection with expression vectors encoding AB_{NFEV}, and the like.

After the cells have been transfected, the transfected or cotransfected cells are incubated with an analyte being tested for ability to antagonize sortilin's effect on the interaction with Aβ peptide. The analyte is assessed for an effect on sortilin transfected or cotransfected cells that is minimal or absent in the negative control cells. In general, the analyte is added to the cell medium the day after the transfection and the cells incubated for one to 24 hours with the analyte. In particular embodiments, the analyte is serially diluted and each dilution provided to a culture of the transfected or cotransfected cells. After the cells have been incubated with the analyte, the medium is removed from the cells and assayed for Aβ. Antibodies specific for each of the metabolites is used to detect the metabolites in the medium. Preferably, the cells are also assessed for viability.

Analytes that alter the accumulation of Aβ in cells, that result in the disappearance of Aβ from the medium, or that effectuate the accumulation of Aβ on the cell surface in the presence of sortilin protein are considered to be modulators of sortilin and are potentially useful as therapeutic agents for sortilin-related diseases including AD. Without wishing to be bound by any theory, it is believed based on the findings herein that sortilin activity will reduce the amount of Aβ in the cell culture medium by internalizing Aβ into the cells. Conversely, reduced sortilin activity, i.e. analytes that inhibits sortilin, will cause retention of Aβ in the medium. Direct inhibition or modulation of sortilin can be confirmed using binding assays using the full-length sortilin, or a domain thereof, or a sortilin fusion protein comprising domain(s) coupled to a C-terminal FLAG, or other, epitopes. A cell-free binding assay using full-length sortilin, or domain(s) thereof, a sortilin fusion protein, or membranes containing sortilin integrated therein and labeled-analyte can be performed by known methods and the amount of labeled analyte bound to sortilin determined.

The present invention further provides a method for measuring the ability of an analyte to modulate the level of sortilin mRNA or protein in a cell. In this method, a cell that expresses sortilin is contacted with a candidate compound and the amount of sortilin mRNA or protein in the cell is determined. This determination of sortilin levels may be made using any of the above-described immunoassays or techniques disclosed herein. The cell can be any sortilin expressing cell such as cell transfected with an expression vector comprising sortilin operably linked to its native promoter or a cell taken from a brain tissue biopsy from a patient.

The present invention further provides a method of determining whether an individual has a sortilin-associated disorder or a predisposition for a sortilin-associated disorder. The method includes providing a tissue or serum sample from an individual and measuring the amount of sortilin in the tissue sample. The amount of sortilin in the sample is then compared to the amount of sortilin in a control sample. An alteration in the amount of sortilin in the sample relative to the amount of sortilin in the control sample indicates the subject has a sortilin-associated disorder. A control sample is preferably taken from a matched individual, that is, an individual of similar age, sex, or other general condition but who is not suspected of having a sortilin related disorder. In another aspect, the control sample may be taken from the subject at a time when the subject is not suspected of having a condition or disorder associated with abnormal expression of sortilin.

Other methods for identifying inhibitors of sortilin can include blocking the interaction between sortilin and Aβ processing or trafficking using standard methodologies for analyzing protein-protein interaction such as fluorescence resonance energy transfer or scintillation proximity assay. Surface Plasmon Resonance can be used to identify molecules that physically interact with purified or recombinant sortilin.

In accordance with yet another embodiment of the present invention, there are provided antibodies having specific affinity for the sortilin or epitope thereof. The term “antibodies” is intended to be a generic term which includes polyclonal antibodies, monoclonal antibodies, Fab fragments, single V_{H} chain antibodies such as those derived from a library of camel or llama antibodies or camelized antibodies (Nuttall et al., Curr. Pharm. Biotechnol. 1:253-263 (2000); Muyldermans, J. Biotechnol. 74:277-302 (2001)), and recombinant antibodies. The term “recombinant antibodies” is intended to be a generic term which includes single polypeptide chains comprising the polypeptide sequence of a whole heavy chain antibody or only the amino terminal variable domain of the single heavy chain antibody (V_{H} chain polypeptides) and single polypeptide chains comprising the variable light chain domain (V_{L}) linked to the variable heavy chain domain (V_{H}) to provide a single recombinant polypeptide comprising the Fv region of the antibody molecule (scFv polypeptides) (see Schmiedl et al., J. Immunol. Meth. 242:101-114 (2000); Schultz et al., Cancer Res. 60: 6663-6669 (2000); Duibel et al., J. Immunol. Meth. 178:201-209 (1995); and in U.S. Pat. No. 6,207,804 B1 to Huston et al.). Construction of recombinant single V_{H} chain or scFv polypeptides which are specific against an analyte can be obtained using currently available molecular techniques such as phage display (de Haard et al., J. Biol. Chem. 274: 18218-18230 (1999); Saviranta et al., Bioconjugate 9:725-735 (1999); de Greeff et al., Infect. Immun. 68: 3949-3955 (2000)) or polypeptide synthesis. In further embodiments, the recombinant antibodies include modifications such as polypeptides having particular amino acid residues or ligands or labels such as horseradish peroxidase, alkaline phosphatase, fluors, and the like. Further still embodiments include fusion polypeptides which comprise the above polypeptides fused to a second polypeptide such as a polypeptide comprising protein A or G.

The antibodies specific for sortilin can be produced by methods known in the art. For example, polyclonal and monoclonal antibodies can be produced by methods well known in the art, as described, for example, in Harlow and Lane, Antibodies: A Laboratory Manual. Cold Spring Harbor Laboratory Press: Cold Spring Harbor, N.Y. (1988). Sortilin or fragments thereof can be used as immunogens for generating such antibodies. Alternatively, synthetic peptides can be prepared (using commercially available synthesizers) and used as immunogens. Amino acid sequences can be analyzed by methods well known in the art to determine whether they encode hydrophobic or hydrophilic domains of the corresponding polypeptide. Altered antibodies such as chimeric, humanized, camelized, CDR-grafted, or bifunctional antibodies can also be produced by methods well known in the art. Such antibodies can also be produced by hybridoma, chemical synthesis or recombinant methods described, for example, in Sambrook et al., supra., and Harlow and Lane, supra. Both anti-peptide and anti-fusion protein antibodies can be used (see, for example, Bahouth et al., Trends Pharmacol. Sci. 12:338 (1991); Ausubel et al., Current Protocols in Molecular Biology, (John Wiley and Sons, N.Y. (1989)).

Antibodies so produced can be used for the immunoaffinity or affinity chromatography purification of sortilin or sortilin/ligand or analyte complexes. The above referenced anti-sortilin antibodies can also be used to modulate the activity of the sortilin in living animals, in humans, or in biological tissues isolated therefrom. Accordingly, contemplated herein are compositions comprising a carrier and an amount of an antibody having specificity for sortilin effective to block naturally occurring sortilin from binding its ligand or for effecting the processing of AB to Aβ peptide.

Therefore, in another aspect, the present invention further provides pharmaceutical compositions that antagonize sortilin's effect on the interaction with Aβ peptide. Such compositions include a sortilin nucleic acid, sortilin peptide, fusion protein comprising sortilin or fragment thereof coupled to a heterologous peptide or protein or fragment thereof, an antibody specific for sortilin, nucleic acid or protein aptamers, siRNA inhibitory to sortilin mRNA, analyte that is a sortilin antagonist, or combinations thereof, and a pharmaceutically acceptable carrier or diluent.

In a further still aspect, the present invention further provides a kit for in vitro diagnosis of disease by detection of sortilin in a biological sample from a patient. A kit for detecting sortilin preferably includes a primary antibody capable of binding to sortilin; and a secondary antibody conjugated to a signal-producing label, the secondary antibody being capable of binding an epitope different from, i.e., spaced from, that to which the primary antibody binds. Such antibodies can be prepared by methods well-known in the art. This kit is most suitable for carrying out a two-antibody sandwich immunoassay, e.g., two-antibody sandwich ELISA.

Using derivatives of sortilin protein or cDNA, dominant negative forms of sortilin that could interfere with sortilin-mediated AB processing to Aβ release can be identified. These derivatives could be used in gene therapy strategies or as protein-based therapies top block sortilin activity in afflicted patients. sortilin can be used to identify endogenous brain proteins that bind to sortilin using biochemical purification, genetic interaction, or other techniques common to those skilled in the art. These proteins or their derivatives can subsequently be used to inhibit sortilin activity and thus be used to treat Alzheimer's disease. Additionally, polymorphisms in the sortilin RNA or in the genomic DNA in and around sortilin could be used to diagnose patients at risk for Alzheimer's disease or to identify likely responders in clinical trials.

The following examples are intended to promote a further understanding of the present invention.

### Example 1

### Rat Primary Hippocampal Neuron Immunofluorescence

Primary hippocampal cultures were prepared from frozen dissociated neonatal rat hippocampal cells (Cambrex, Corp., East Rutherford, N.J.) that were thawed and plated in 96-well plates (Costar, Corning Life Science, Corning N.Y.) at a concentration of 20,000 cells per well (plated at Analytical Biological Services Inc., Wilmington Del.). The cells were maintained in media (Neurobasal without L-glutamine, supplemented with B27, Gibco, Carlsbad, Calif.) for a period of two weeks and then used for binding studies. Primary hippocampal neurons (cultured for 14 days) were incubated with 5-25 μM ADDLs or bADDLs (bADDLs are ADDLs made with biotinylated Aβ42, a modification of methods described in Lambert M P, et al., Proc Natl Acad Sci USA 95(11):6448 (1998)) for one hour at 37° C. and then the cells washed 3-4 times with warm culture media to remove unbound ADDLs or bADDLs. The cells were then fixed with 4% paraformaldehyde solution for ten minutes at room temperature (RT), the solution removed and fresh fixative added for an additional ten minutes at RT. The cells were then permeabilized (4% paraformaldehyde solution with 0.1% triton-X 100, Sigma, St. Louis Mo.) for ten minutes, washed six times with PBS and then incubated for one hour at 37° C. with blocking buffer (PBS with 10% Bovine Serum Albumin, BSA; Sigma A-4503, St. Louis, Mo.). To detect ADDL binding the cells were incubated overnight at 37° C. with 4G8 (Signet Labs Princeton, N.J., diluted 1:1,000 in PBS containing 1% BSA) to detect tau, and 6E10 (Signet Labs, Princeton, N.J.; 1:1,000) to detect ADDLs. In addition, a polyclonal antiserum raised against tau (Sigma, 1:1,000, St. Louis, Mo.) was used to visualize the cell processes. The next day, the cells were washed three times with PBS, incubated for one hour at room temperature with an Alexa 594-labeled anti-mouse secondary (Molecular Probes diluted 1:500 in PBS with 1% BSA, Eugene, Oreg.) and an Alexa 488-labeled anti-rabbit secondary (Molecular Probes, diluted 1:1,000, Eugene, Oreg.), washed three times in PBS and then the binding observed using a microscope with fluorescence capabilities.

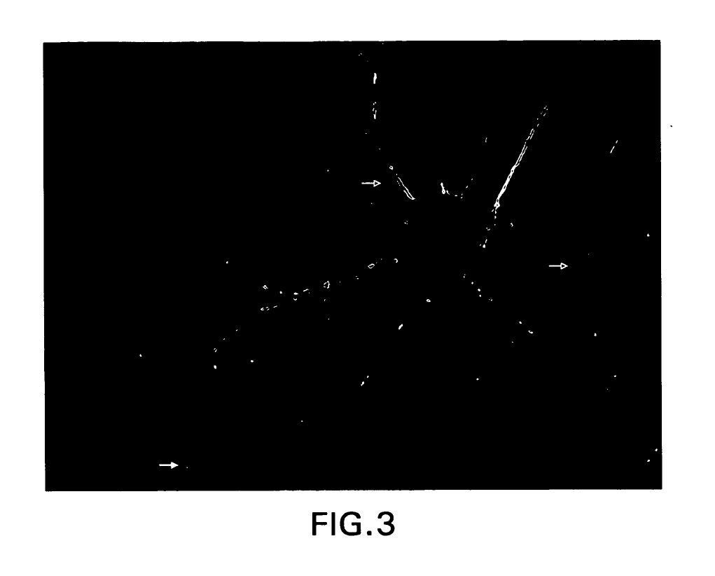

Results from this experiment are shown in FIG. 3. The staining pattern of ADDLs is denoted by arrows and is consistent with the punctate, cell surface-associated pattern typically associated with a ligand-receptor interaction. The adjacent cells are not stained and show the cell-type specificity of this ADDL staining pattern and serve also as a negative internal control against non-specific binding. This data supports the possibility that receptor(s) for ADDLs exists in hippocampal neurons, as previously suggested (Lambert M P, et al., Proc Natl Acad Sci USA 95(11):6448-53 (1998)).

### Example 2

### Sortilin as ADDL Receptor

This example describes the identification of sortilin as a receptor for ADDLs. A schematic overview of the experiment is shown in FIG. 4A. Thirty male Spraque Dawley rats were ordered from Taconic Farms (Germantown, N.Y.) for this experiment, weighing between 250 g and 300 g. Rats were sacrificed, the brain was removed and the hippocampus and cerebellum were collected in lysis buffer. Equivalent tissue weights of hippocampi and cerebellum (2.21 g of each) were isolated and homogenized in 10 ml lysis buffer (15 mM NaCl2, 2 mM MgCl2, 10 mM HEPES, 1 mM sodium orthovanidate, and protease inhibitors (Complete tablets, EDTA free). The hippocampus and cerebellum were dounce homogenized for about 25 strokes until the cells were broken and nuclei could be seen in the homogenate microscopically. The homogenate was then spun ten minutes at 1000×g two times to remove nuclei and organelles. The supernatant (supt) was collected and spun at 100,000×g for one hour. The pellet was resuspended in 2 ml of F12 with 1% NP40 and 0.1% Triton X-100. The membrane preparations were sonicated briefly on ice to resuspend the pellet. A BCA assay was performed in order to determine protein concentration before pre-clear to normalize; both samples had equivalent protein concentration. Pre-clear was performed using 100 μl/ml streptavidin (SA) beads two times for 30 minutes. After pre-clearance, 20 ml of b(EV) ADDL 1-42 was added to 5 ml of each pre-cleared supernatant and allowed to bind overnight at 4° C. b(EV)ADDL1-42 is an oligomeric species of Aβ42 that differs from endogenous Aβ42 by the substitution of EV for DA at the first two amino acid positions. Bound bADDL was cross-linked with Sulfo-EGS (EGS: ethylene glycol-bis-sulfosuccinimidyl succinate) (Pierce, Rockford, Ill.) at 1 mM final concentration for two hours at 4° C. Reaction was quenched with 1M Tris pH 7.5. SA beads were added at 100 μl/ml to capture cross-linked receptor. Beads were pelleted and washed three times with high salt wash, a OD₂₈₀ was taken to measure the degree of clearance of nonspecifically bound protein. Amine bond was broken with hydroxylamine HCl at 37° C. for three hours. Beads were pelleted and 3 ml of beads were resuspended with 1 ml of sample buffer, all samples were diluted with sample buffer, denatured for five minutes at 95° C. and frozen. 4-20% Tris-HCl 12-well gels were run and sections were analyzed by MS/MS (FIG. 4B). One of the proteins recovered with ADDLs from the hippocampus (lane 4), but not the cerebellum (lane 5), where AD pathology is much reduced in humans, was identified as sortilin. Sortilin was confirmed by western blot with anti-sortilin antibodies in the same samples (C—cerebellum, H—hippocampus, Brain Hmgt—unpurified brain homogenate used in the experiment, Supt—supernatant from the bADDL pull down experiment, pellet—proteins recovered with the streptavidin beads, kD—estimated molecular weight in kilodaltons), and was further shown to be abundantly expressed as multiple species in B103 neuroblastoma cells relative to CHO fibroblasts (FIG. 4C).

### Example 3

### Immunoprecipitation with bADDLs

A 6-well tissue culture plate was planted with 500,000 cells/well and transfected with sortilin cDNA the next day using lipofectamine 2000 (Invitrogen, Carlsbad Calif.). The transfection was allowed to go for 48 hours at 37° C. 5% CO₂ and the cells were harvested with co-immunoprecipitation buffer (CO—IP) Tris-HCl pH 7.5, NaCl₂, NP40, protease inhibitors. Conditioned media from transfection was also collected. Lysate and conditioned media were pre-cleared with SA beads three times for two hours and then 8 μM bADDL 1-40 and 1-42 were added and allowed to bind overnight at 4° C. with rocking. The next day anti-sortilin antibody and protein A beads were added to the tubes and spun down, the beads were washed three times with buffer and the pellet was resuspended in equivalent amount of 2× sample buffer and boiled at 95° C. for five minutes. A 4-20% Tris-HCl Criterion gel was run and transferred, a western was performed with anti-sortilin antibody to visualize the immunoprecipitated sortilin; alternatively 6E10 (Signet Labs, Princeton, N.J.) antibody was used to visualize Aβ species. FIG. 5 shows the physical interaction between sortilin and Aβ monomers, dimers and other species. Lane 1 shows that no sortilin or Aβ was recovered if anti-sortilin was omitted and serves as a specificity control. Lanes 2 and 3 show the amount of exogenous A040 or Aβ42 (ex ADDL) recovered with sortilin antibodies. This data confirmed that sortilin and Aβ monomers and oligomers exist in complex in tissue culture media, which further supports the invention herein of the use of sortilin as a receptor for Aβ.

### Example 4

### Localization of Sortilin with Amyloid Plaques

Immunohistochemistry of mouse brain slices was performed using standard methods, as detailed below, to show the localization of sortilin with amyloid plaques in transgenic mice.

Wash buffer was prepared at a 1:20 dilution in sterile water (BioGenex, San Ramon, Calif.). Slides of sagital section of preserved mouse brain were placed in following solutions in a Tissue Tek II for 2-3 minutes each: Xylene1 (HistoPrep, Fisher, Waltham Mass.), Xylene 2, 100% Ethanol, 95% Ethanol, 70% Ethanol, and tap water, then placed slides in wash buffer. Slides were placed in a container filled to the top with Antigen Retrieval Citra (BioGenex, San Ramon, Calif.). The container was placed in microwave and heated for desired amount of time on power level J. Immediately after microwaving slides were placed in the sink and cold tap water was flowed into the container for 2-3 minutes. Slides were placed into 200 ml of 0.3% hydrogen peroxide for 30 minutes before washing 2-3 times in wash buffer. In an incubation chamber at room temperature 500 μl of 5% serum was added to slides and incubated for 15 minutes. The slides were dried and 500 μl of primary antibody dilutions were added. After overnight incubation at 4° C., the next day solution was drained and the slides washed three times, 1:200 dilution of biotinylated secondary antibody was added to each slide and incubated for 30 minutes. After washing, antibody-antigen complexes were visualized using Vectastain ABC following the manufacturer's recommendations (Vector Laboratories, Peterborough UK). FIG. 6 shows the results of this experiment in aged Tg2576 mice, which accumulate Aβ into amyloid plaques. Sortilin immunoreactivity was present within the amyloid plaques which is consistent with a physical interaction between sortilin and Aβ in vivo.

### Example 5

### ELISA to Measure Aβ Levels

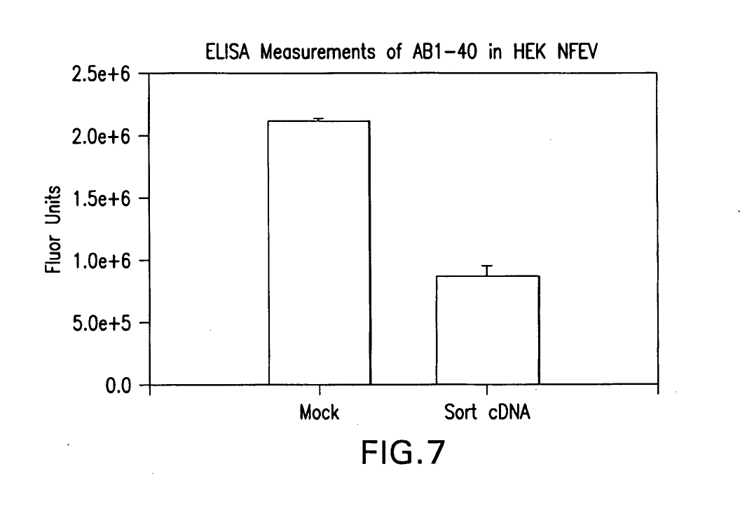

Aβ levels were measured by ELISA using known methodology as described in detail in Majercak J, et al., Proc Natl Acad Sci USA 103(47):17967-17972 (2006). Sortilin cDNA was transfected into HEK293 cells using standard methods. Aβ40 was pipetted into the well, incubated under standard growth conditions in a tissue culture incubator overnight, and then Aβ levels were measured the following day by ELISA. The results of this experiment are shown in FIG. 7. The lower levels of Aβ40 in the wells of cells overexpressing sortilin is consistent with the discovery that sortilin is a receptor for Aβ, because receptor-mediated internalization of Aβ would lead to less Aβ in the tissue culture medium relative to controls.

### Example 6

### Tissue Expression of Sortilin

Because sortilin appeared to be a receptor to Aβ, which has a known role in the neuritic plaques associated with Alzheimer's disease, expression of sortilin was examined in a variety of tissues to determine whether sortilin was expressed in the brain.

A proprietary database, the TGI Body Atlas, was used to show that the results of a microarray analysis of the expression of a majority of characterized genes, including sortilin, in the human genome in a panel of different tissues. Sortilin mRNA was found to be expressed predominantly in the brain and within cortical structures such as the temporal lobe, entorhinal cortex, and frontal cortex, all of which are subjected to amyloid Aβ deposition and Alzheimer pathology. The results are shown graphically in FIG. 4.

The results of this example reinforce the conclusions drawn from Examples 1-5 in that those skilled in the art would expect that a physiologically relevant receptor for Aβ would be expressed preferentially in the brain, the site at which most Aβ is generated and where Aβ toxicity is known to occur.

### Example 7

### Identification of Analytes that Modulate Sortilin

The results of Examples 1-6 have shown that sortilin is a receptor for Aβ, which has a role in the pathology of Alzheimer's disease. This suggests that analytes that antagonize sortilin interaction with Aβ will be useful for the treatment or therapy of Alzheimer's disease. Therefore, there is a need for assays to identify analytes that modify sortilin's activity, for example, that bind to and neutralize sortilin's interaction with Aβ. The following is an assay that can be used to identify analytes that modulate sortilin's activity.

A screen for sortilin-derived agents that bind and neutralize Aβ, for therapeutic use in AD, can be performed in which sortilin, or fragments derived from sortilin, are tested for the ability to block Aβ toxicity in a model neuronal system. Aβ42 is allowed to aggregate into a toxic species as is known in the art. See for example, the use of cytotoxic amyloid peptides that inhibit cellular 3-(4,5-dimethylthiazol-2-yl)-2,5-diphenyltetrazolium bromide (MTT) reduction by enhancing MTT formazan exocytosis., Y. Liu and D. Schubert, J. Neurochem. 69:2285-2293, (1997). The soluble N-terminus of the sortilin receptor is expressed by cloning the cDNA minus the transmembrane domain and cytoplasmic tail into an appropriate expression vector and transfecting into a mammalian cell line that secretes quantities of sortilin. Sortilin soluble N-terminal domain will be collected using immunoprecipitation with sortilin antibodies to the N-terminus (for example Becton-Dickinson, Franklin Lakes, N.J.) and eluted by acid and neutralized. The sortilin fragment once added to the culture medium of PC12 cells binds the toxic Aβ and prevents the activation of apoptosis. This effect is measured by adding a single concentration of Aβ to a 96-well assay plate containing 10,000 PC-12 cells/well. Cells are incubated at 37° C.+5% CO₂ overnight. The next day, toxicity is monitored by measuring activity of the apoptotic marker Caspase 3 (Promega, Madison, Wis., CaspACE Assay System, Colorimetric). Cell monolayers are washed with ice-cold PBS, and resuspended in the provided Cell Lysis Buffer. Lysate is centrifuged and the supernatant is used to assay for caspase activity. Two μl of substrate is added to each lysate sample, the plate is covered and incubated at 37° C. for four hours. The plate is measured in the spectrophotometer for absorbance at 405 nM. Caspase specific activity is determined by subtracting the sortilin minus N-terminal binding domain from the full length titration. Fragments of sortilin, used either alone or complexed with another protein (such as a part of an IgG protein) are assayed the same way.

### Example 8

### Identification of Analytes that Block Sortilin-Aβ Interaction

In another embodiment of the invention described herein, a screen can be performed to identify therapeutic agents for the treatment of AD that block the sortilin-Aβ interaction and, as such, prevent Aβ toxicity to neurons. In this embodiment agents are evaluated for their ability to repress Aβ-mediated caspase activation in PC12 cells as above, but without the addition of sortilin or its fragments into the medium. Agents identified that repress the toxicity of Aβ as measured in this assay are confirmed to be specific to sortilin.

To confirm direct inhibition or modulation of sortilin, the sortilin extracellular domain is subcloned into vectors such that a fusion protein with C-terminal FLAG epitopes are encoded. Protein constructs are purified by affinity chromatography, according to manufacturer's instructions, using an ANTI-FLAG M2 agarose resin. Sortilin constructs are eluted from the ANTI-FLAG column by the addition of FLAG peptide (Asp-Tyr-Lys-Asp-Asp-Asp-Asp-Lys, SEQ ID NO: 4) (Sigma, St. Louis, Mo.) resuspended in TBS (50 mM Tris HCl pH 7.4, 150 mM NaCl) to 100 μg/ml. Fractions are collected and concentrations are determined by A280. A PD-1 column (Amersham, Little Chalfont. UK) is used to buffer exchange all eluted fractions containing the protein of interest and simultaneously remove excess FLAG peptide. The FLAG-sortilin constructs are conjugated to the S series CM5 chip surface (Biacore, Piscataway, N.J.) using amine coupling as directed by the manufacturer. A pH scouting protocol is used to determine the optimal pH conditions for immobilization. Immobilization is conducted at an empirically determined temperature in PBS pH 7.4 or another similar buffer following a standard Biacore immobilization protocol (Biacore, Piscataway, N.J.). The reference spot on the CM5 chip (Biacore, Piscataway, N.J.) (a non-immobilized surface) will serve as background. The third spot on the CM5 chip is conjugated with bovine serum albumin in a similar fashion to serve as a specificity control. Interaction of the putative sortilin modulator at various concentrations and sortilin are analyzed using the compound characterization wizard on the Biacore S51 (Biacore, Piscataway, N.J.). Binding experiments are completed at 30° C. using 50 mM Tris pH 7, 200 uM MnCl2 or MgCl2 (+5% DMSO) or a similar buffer as the running buffer. Prior to each characterization the instrument is equilibrated three times with assay buffer. Default instructions for characterization will be a contact time of 60 seconds, sample injection of 180 seconds and a baseline stabilization of 30 seconds. All solutions are added at a rate of 30 μl/min. Using the BiaEvaluation software (from Biacore, Piscataway, N.J.) each set of sensorgrams derived from the ligand flowing through the sortilin-conjugated sensor chip is evaluated and an affinity constant, if binding is observed, is determined.

### Example 9

### Screening for Modulators of Aβ Toxicity

The discovery herein that sortilin is a receptor for Aβ enables screening for other molecules that modulate Aβ toxicity that can be used as therapeutic agents to treat or diagnose AD. 100 mg of frozen human brain tissue (cortex or hippocampus) is obtained from an appropriate vendor and solubilized in 10 volumes of 50 mM Tris pH 8.0, 1% NP-40, 150 mM NaCl, and 0.5% Triton X-100 by dounce homogenization. Insoluble material is removed by centrifugation and the supernatant is incubated overnight at 4° C. with 100 μL of M2 anti-FLAG resin (see above) to clear proteins that interact non-specifically with that reagent. After centrifugation the supernatant is incubated with 100 μL of M2 anti-Flag resin plus 100 μg of the FLAG-sortilin fusion protein used above. After overnight incubation with gentle rocking at 4° C., the bead-antibody-sortilin complex is recovered by centrifugation and washed four times in ten volumes of lysis buffer (same buffer as above). Sortilin-FLAG and co-purifying proteins is by adding FLAG peptide as above, then denatured in 2% SDS and analyzed by SDS-PAGE followed by silver staining (Bio-Rad, Foster City, Calif.). Proteins that co-purify with sortilin are excised from the SDS-PAGE gel, digested by trypsin, and identified by mass spectrometry followed by database searching using the same methods used to identify sortilin.

The proteins that are purified with the FLAG-sortilin construct are assessed for effects on Aβ toxicity. A cDNA for the identified gene is transfected into PC12 cells using lipofectamine 2000, and toxic Aβ added to the cell culture as described above, with the exception that in this instance the exact dose of Aβ needed to produce a 50% toxic effect is administered to the cells. Overexpression of a protein that modulates the toxicity of AB will significantly alter caspase activation, with a pro-toxic protein causing more caspase activation, while an inhibitor of Aβ toxicity causes less caspase activation.

### Example 10

### Reduction of Sortilin Expression as a Therapeutic Treatment for AD

This example describes a method to reduce sortilin expression to provide therapeutic benefit to a patient with Alzheimer's disease. siRNA molecules targeting sortilin mRNA (both rodent and primate) are synthesized and transfected into HEK293 cells using Lipofectamine 2000 following standard protocols known in the art. Sortilin RNA levels are then measured 24 hours later using quantitative real-time polymerase chain reaction using sequence specific primers and probe using standard methodologies available from Applied Biosystems, Inc. (Foster City, Calif.). siRNAs that effectively reduce sortilin RNA, but not RNAs for control genes, are thereby identified and injected into the brain of a test organism such as a mouse to establish doses of siRNAs that reduce sortilin RNA in the central nervous system (as measured by real-time PCR as above, except from whole brain RNA). These siRNAs would be used to reduce sortilin expression, and thus Aβ internalization, in AD patients.

### Example 11

### Polyclonal Antibodies Specific for Sortilin

This example describes a method for making therapeutic polyclonal antibodies specific for sortilin, a peptide fragment of sortilin, or epitope thereof.

Sortilin is produced as described in Example 1, or a peptide fragment/epitope comprising a particular amino acid sequence of sortilin is synthesized, and coupled to a carrier such as BSA or KLH. Antibodies are generated in New Zealand white rabbits over a 10-week period. The sortilin, peptide fragment or epitope is emulsified by mixing with an equal volume of Freund's complete adjuvant and injected into three subcutaneous dorsal sites for a total of about 0.1 mg sortilin per immunization. A booster containing about 0.1 mg sortilin (or peptide fragment/epitope) emulsified in an equal volume of Freund's incomplete adjuvant is administered subcutaneously two weeks later. Animals are bled from the articular artery. The blood is allowed to clot and the serum collected by centrifugation. The serum is stored at −20° C.

For purification, the sortilin is immobilized on an activated support. Antisera is passed through the sera column and then washed. Specific antibodies are eluted via a pH gradient, collected, and stored in a borate buffer (0.125M total borate) at 0.25 mg/mL. The anti-sortilin antibody titers are determined using ELISA methodology with free sortilin bound in solid phase (1 pg/well). Detection is obtained using biotinylated anti-rabbit IgG, HRP-SA conjugate, and ABTS. The purified anti-sortilin antibodies are then tested for ability to interfere with the ability of sortilin to bind Aβ using either of the methods described above.

### Example 12

### Monoclonal Antibodies Specific for Sortilin

This example describes a method for making monoclonal antibodies specific for sortilin.

BALB/c mice are immunized with an initial injection of about 1 μg of purified sortilin per mouse mixed 1:1 with Freund's complete adjuvant. After two weeks, a booster injection of about 1 μg of the antigen is injected into each mouse intravenously without adjuvant. Three days after the booster injection serum from each of the mice is checked for antibodies specific for the sortilin.

The spleens are removed from mice positive for antibodies specific for the sortilin and washed three times with serum-free DMEM and placed in a sterile Petri dish containing about 20 mL of DMEM containing 20% fetal bovine serum, 1 mM pyruvate, 100 units penicillin, and 100 units streptomycin. The cells are released by perfusion with a 23 gauge needle. Afterwards, the cells are pelleted by low-speed centrifugation and the cell pellet is resuspended in 5 mL 0.17 M ammonium chloride and placed on ice for several minutes. Then 5 mL of 20% bovine fetal serum is added and the cells pelleted by low-speed centrifugation. The cells are then resuspended in 10 mL DMEM and mixed with mid-log phase myeloma cells in serum-free DMEM to give a ratio of 3:1. The cell mixture is pelleted by low-speed centrifugation, the supernatant fraction removed, and the pellet allowed to stand for 5 minutes. Next, over a period of 1 minute, 1 mL of 50% polyethylene glycol (PEG) in 0.01 M HEPES, pH 8.1, at 37° C. is added. After 1 minute incubation at 37° C., 1 mL of DMEM is added for a period of another 1 minute, then a third addition of DMEM is added for a further period of 1 minute. Finally, 10 mL of DMEM is added over a period of 2 minutes. Afterwards, the cells are pelleted by low-speed centrifugation and the pellet resuspended in DMEM containing 20% fetal bovine serum, 0.016 mM thymidine, 0.1 hypoxanthine, 0.5 μM aminopterin, and 10% hybridoma cloning factor (HAT medium). The cells are then plated into 96-well plates.

After 3, 5, and 7 days, half the medium in the plates is removed and replaced with fresh HAT medium. After 11 days, the hybridoma cell supernatant is screened by an ELISA assay. In this assay, 96-well plates are coated with the sortilin. One hundred μL of supernatant from each well is added to a corresponding well on a screening plate and incubated for 1 hour at room temperature. After incubation, each well is washed three times with water and 100 μL of a horseradish peroxide conjugate of goat anti-mouse IgG (H+L), A, M (1:1,500 dilution) is added to each well and incubated for 1 hour at room temperature. Afterwards, the wells are washed three times with water and the substrate OPD/hydrogen peroxide is added and the reaction is allowed to proceed for about 15 minutes at room temperature. Then 100 μL of 1 M HCl is added to stop the reaction and the absorbance of the wells is measured at 490 nm. Cultures that have an absorbance greater than the control wells are removed to two cm² culture dishes, with the addition of normal mouse spleen cells in HAT medium. After a further three days, the cultures are re-screened as above and those that are positive are cloned by limiting dilution. The cells in each two cm² culture dish are counted and the cell concentration adjusted to 1×10⁵ cells per mL. The cells are diluted in complete medium and normal mouse spleen cells are added. The cells are plated in 96-well plates for each dilution. After 10 days, the cells are screened for growth. The growth positive wells are screened for antibody production; those testing positive are expanded to 2 cm² cultures and provided with normal mouse spleen cells. This cloning procedure is repeated until stable antibody producing hybridomas are obtained. The stable hybridomas are progressively expanded to larger culture dishes to provide stocks of the cells.

Production of ascites fluid is performed by injecting intraperitoneally 0.5 mL of pristane into female mice to prime the mice for ascites production. After 10 to 60 days, 4.5×10⁶ cells are injected intraperitoneally into each mouse and ascites fluid is harvested between 7 and 14 days later.

The purified anti-sortilin antibodies are then tested for ability to interfere with the ability of sortilin to bind Aβ using the methods described above.

While the present invention is described herein with reference to illustrated embodiments, it should be understood that the invention is not limited hereto. Those having ordinary skill in the art and access to the teachings herein will recognize additional modifications and embodiments within the scope thereof. Therefore, the present invention is limited only by the claims attached herein.

1. A method for screening for analytes that modulate the interaction of sortilin and Aβ peptide, comprising:
(a) incubating cells, sensitive to the toxic effects of Aβ or that bind Aβ, in a culture medium with soluble sortilin under conditions for expression of the sortilin and said cells;
(b) adding an analyte to said culture medium; and
(c) measuring the level of cytotoxicity or Aβ binding in said cells;
wherein a change in the level of cytotoxicity or Aβ binding indicates that the analyte is an modulator of the interaction of sortilin and Aβ peptide.

2. A method of claim 1 further comprising adding Aβ with an analyte to said culture medium and wherein a change in the level of cytotoxicity or Aβ binding indicates that the analyte is a modulator of the interaction of sortilin and Aβ peptide.

3. A method of claim 1 wherein a decrease in the amount of cytotoxicity or Aβ binding indicates that the analyte is an antagonist of sortilin.

4. A method of claim 1 wherein said cells each comprise a first nucleic acid that encodes the secreted extracellular domain of sortilin operably linked to a first heterologous promoter.

5. The method of claim 4 wherein a control is provided which comprises providing recombinant cells which do not express sortilin.

6. A method for treating Alzheimer's disease in an individual comprising providing to the individual an effective amount of an antagonist of sortilin activity.

7. A method for identifying an individual who has Alzheimer's disease or is at risk of developing Alzheimer's disease comprising obtaining a sample from the individual and measuring the amount of sortilin in the sample.
